(19) United States
(12) Patent Application Publication
(10) Pub. No.: US 2020/0060770 A1
(43) Pub. Date: Feb. 27, 2020

(54) ELECTRODE LEAD WITH INTEGRATED DEFORMATION SENSOR

(71) Applicant: BIOTRONIK SE & Co. KG, Berlin (DE)

(72) Inventor: André van Ooyen, Berlin (DE)

(21) Appl. No.: 16/534,084

(22) Filed: Aug. 7, 2019

(30) Foreign Application Priority Data
Aug. 24, 2018 (DE) 10 2018 120 761.1

(51) Int. Cl. A61B 34/20; A61B 5/042; A61N 1/37

(52) U.S. Cl. CPC A61B 34/20; A61B 2034/2061; A61N 1/37; A61B 5/042

(57) ABSTRACT

An implantable electrode lead with a preferably elongate electrode lead body, which is equipped with a first electrical conductor, and a second electrical conductor, wherein the first electrical conductor and the second electrical conductor are arranged in an interior area of the electrode lead body. The electrode lead body has a deformation sensor element arranged along the longitudinal axis of the electrode lead body, wherein the first electrical conductor and the second electrical conductor are conductively connected to the deformation sensor element, and wherein the deformation sensor element comprises a biocompatible elastomer enriched with conductive particles.

### CROSS-REFERENCE TO RELATED APPLICATIONS

This patent application claims the benefit of and priority to co-pending German Patent Application No. DE 10 2018 120 761.1 filed on Aug. 24, 2018 in the German Patent Office, which is hereby incorporated by reference in its entirety.

### TECHNICAL FIELD

The present invention relates to an electrode lead for a functional electrical stimulation device (FES) with which deformations of the electrode lead body are detectable.

### BACKGROUND

Electrode leads for medical applications are preferably used as parts of an implantable functional electrical stimulation device (FES) for electrical treatment of nerve or muscle cells in the diagnostic or therapeutic field.

Implantable functional electrical stimulation devices, for example, include conventional cardiac pacemakers with a pulse generator for artificially exciting a heartbeat for anti-bradycardia therapy, 3-chamber pacemakers for cardiac resynchronization therapy (CRT) in the case of asynchronous contraction of the cardiac muscle, usually within the scope of a left bundle branch block, and at the same time highly limited pumping function with symptomatic progression, or implantable cardioverters/defibrillators (ICDs) for the treatment of life-threatening cardiac arrhythmias by the delivery of shocks for stopping ventricular fibrillations or for stopping ineffective overstimulation, and by overstimulation (ATP=anti-tachycardia pacing) for stopping stable ventricular tachycardias.

These electrical stimulation devices usually comprise a housing that is compatible with the body and which has an associated electronic circuit and a power supply, for example, a battery. The housing has at least one connection point, to which the electrode lead or the electrode leads can be connected. The electrode leads are sometimes also referred to as probes. The electrode leads are used to transmit the electrical energy from the housing to the tissue/body part to be treated, and vice versa.

Here, the term “electrode lead” in the field of medical technology means not only an element with which electrical energy is transmitted in accordance with the physical definition, but also includes a lead with an electrical conductor together with its encasing insulation, and all further functional elements which are fixedly connected to the lead.

An electrode lead of this kind consists of an elongate body (lead body or electrode lead body), which consists of an insulating material, typically in the form of an insulation or multi-lumen tube formed from an elastomer (generally from silicone or polyurethane) and within which the electrical conductors run along the lead body. The lead body has a proximal end and a distal end. At least one male connector is situated at the proximal end of the lead body and can be connected to a connector in the connection housing of the implant—generally a female connector. The male connector is usually standardized and, for example, in the field of implants for electrical therapy of the heart can be designed in accordance with one of the standards IS-1, IS-4, DF-1 or DF-4. Each of the electrically active contacts of the plug is electrically connected to an electrical feed line, which is in turn electrically attached at or in the vicinity of the distal end of the lead body, generally to an electrically active surface (also referred to as an “electrode pole” or “electrode”), which lies at or in the vicinity of the distal end. Each of these connection lines is guided in an insulated manner. The electrically active surfaces are used to induce electrical therapy at the body part to be treated, for example, in or at the heart, and/or to record measurement signals for diagnostic purposes.

In particular, the electrode poles can be embodied in the form of an electrode tip, a plurality of ring electrodes, or a plurality of electrode coils (shock coils). An electrode tip is often also provided with anchoring elements or retaining structures, by means of which the constancy of the spatial position of the point of transfer of the electrical energy in the tissue to be treated can be ensured. The electrode poles, which form the points of transfer of the electrical energy into the tissue, can be designed as pickup electrodes, stimulation electrodes or measurement electrodes.

A cardiac pacemaker or cardioverter/defibrillator typically uses the intracardiac ECG measurement (IEGM) to determine the intrinsic excitation of the heart in the areas of heart provided with electrode leads, so as to thus obtain a starting point for the time and as applicable the intensity of the contraction of the heart. The intensity and time delay with which the myocardium responds to the intrinsic excitation of the heart or to a stimulation pulse of a cardiac pacemaker is primarily dependent on the state of health of the heart tissue. A comparison of the electrical excitation (intrinsic or by stimulation) of the heart with the mechanical contraction of the myocardium thus makes it possible to draw conclusions regarding the state of the myocardium.

The present invention is directed at overcoming one or more of the above-mentioned problems.

### SUMMARY

On this basis, an object is to create a robust, durable and economical electrode lead with which the cardiac activity is detectable alternatively or additionally to the intracardiac ECG measurement.

At least this problem is solved by an electrode lead having the features of claim 1. Further embodiments are specified in the corresponding dependent claims and will be described hereinafter.

Claim 1 provides an implantable electrode lead with a preferably elongate electrode lead body, which extends in particular along a longitudinal axis, and which is equipped with a first electrical conductor and a second electrical conductor, wherein the first electrical conductor and the second electrical conductor are arranged in at least one interior area of the electrode lead body, and a deformation sensor element arranged along the longitudinal axis of the electrode lead body, wherein the first electrical conductor and the second electrical conductor are conductively connected to the deformation sensor element, and wherein the deformation sensor element comprises a biocompatible elastomer enriched with conductive particles.

An electrode lead that is implanted in the human or animal body (referred to hereinafter just as a body), on account of its position in the body, has a three-dimensional profile in space along its longitudinal axis, because the curvature of the electrode lead varies along its longitudinal axis on account of the predefined conditions in the body. If the region of the body into which the electrode lead is implanted moves, the body thus exerts force onto the electrode lead at a specific point, such that the electrode lead is deformed and adapts its profile to the altered form of the body region in which it is implanted. If the deformation of the electrode lead is determined by means of a deformation sensor element, a force effect necessary for this deformation, for example, the movement at a specific point of the body, can thus be detected.

Implantable electrode leads are developed especially for the body regions in which they are to be implanted and are manufactured in various sizes (for example, in various lengths and diameters) for different both sizes and implantation objectives. Thus, it is already known, prior to the implantation of an electrode lead, which region of the electrode lead will come to lie against which body point following the implantation. A person skilled in the art is thus able to find a position along the electrode lead body at which the deformation sensor element should be placed in order to measure a force effect on the electrode lead at a specific point in the body.

In this regard, for example, the course of the electrode lead in the region of the heart is of interest, the implanted electrode lead typically running in the atrium or in the atrium and in the ventricle. As a result of the movement of the heart an electrode lead running there is deformed with each heartbeat. In order to detect this movement, the deformation sensor element must be positioned at a point along the electrode lead body in the region of the atrium and/or in the region of the ventricle. In other regions of the body as well, movements can be detected by electrode leads which are adapted especially to these regions and which have one or more deformation sensor elements.

Due to the deformation sensor element arranged along the longitudinal axis of the electrode lead body, it is possible to detect deformations of the electrode lead in the region of the deformation sensor element. A deformation of the electrode lead in this sense occurs, for example, when the electrode lead is bent, such that it has a different course in space along its longitudinal axis after the bending as compared to before the bending. Alternatively, a deformation of the electrode lead in this sense also occurs when pressure is exerted on part of the electrode lead. The deformation sensor element is thus also suitable for determining the pressure exerted on the electrode lead and, in particular, the exerted pressure profile in the region of the deformation sensor element.

The human heart on average runs through approximately 100,000 cardiac cycles daily and thus performs as many contractions over the course of a day. The described deformation sensor element is thus subjected to a significant mechanical loading. For this reason, it is of great advantage if the deformation sensor element consists of a material of which the mechanical properties are similar to the mechanical properties of the insulation material of the electrode lead body. The insulating part of the electrode lead body is, for example, embodied as an insulation or multi-lumen tube formed from an elastomer which has electrically insulating and elastic properties. Possible materials include: natural and synthetic rubber materials, such as styrene-butadiene rubber (SBR), acrylonitrile butadiene rubber (AB), also referred to as nitrile rubber (NBR=nitrile butadiene rubber) for short, silicone rubber, thermoplastic elastomers and liquid rubber. The following are preferably used: silicone, polyurethane (PU) or polyether block amide block copolymer from the PEBAX® range by Arkema. For this reason, the deformation sensor element likewise comprises a biocompatible elastomer, which is enriched with conductive particles.

The deformation exerted on the deformation sensor element leads to a modification of the geometric positions of the conductive particles embedded in the elastomer relative to one another, which leads to a modification of the conductivity of the deformation sensor element. This change in conductivity is brought about—depending on the embedded conductive particles—by resistive effects or by the quantum mechanical tunnel effect. A deformation exerted on the deformation sensor element can be determined by the measurement of the electrical conductivity of the deformation sensor element.

In order to measure the deformation by means of resistive effects, the elastomer of the deformation sensor element is made conductive extrinsically by the addition of conductive particles. The conductive particles act here as a filler on the elastomer. The conductive particles are intended to create in the elastomer a closed network of continuous article strands, or what is known as a percolation network, which provides the necessary conductive paths. A minimum concentration of the conductive particles is necessary for this purpose. The amount of required conductive particles is heavily dependent on the material of the used conductive particles. The conductivity at the percolation threshold, at which the network is formed for the first time, rises suddenly by orders of magnitude. As a result of the deformation of the elastomer to which conductive particles were added, the percolation network is modified such that more or fewer conductive paths are formed, such that the conductivity of the deformation sensor element thus changes.

If the elastomer is enriched with specific conductive particles, a quantum tunneling composite (QTC) thus forms. In a QTC there is no formation of a continuous percolation network in spite of significant particle concentrations. Rather, the electrical conduction occurs in these materials by tunnel currents on account of the quantum mechanical tunnel effect. The deformation exerted on the deformation sensor element leads to a modification of the geometric positions of the conductive particles embedded in the elastomer relative to one another, whereby the effective conductivity resulting from the individual tunnel currents changes.

In a further embodiment, the proportion of conductive particles in the elastomer to which conductive particles were added is 0.8 vol. % to 45 vol. % (vol. %=volume percent). The suitable volume share of conductive particles in the elastomer to which they were added is dependent on the material and on the geometric form of the particles, which, for example, can be expressed by the aspect ratio (AR for short). The aspect ratios and a range for the volume fractions of conductive particles formed from the below-stated materials in the elastomer to which said conductive particles were added are specified here by way of example: conductive carbon, such as conductive carbon blacks (AR=1.7, 5-30 vol. %) or carbon fibers (AR=16, 3-10 vol. %), metal-coated graphite (for example coated with nickel, AR=2, 6-15 vol. %) and metal-coated carbon (for example, coated with nickel), single-walled nanotubes (SWNT, AR=1000, 0.8-3.0 vol. %)s or multi-walled nanotubes (MWNT) or nickel powder (AR approximately 1, 30-45 vol. %).

Within the scope of the present disclosure, in particular, the proximal end or the proximal portion of the electrode lead body is the end or the region by means of which the electrode lead body is connected to a functional electrical stimulation device. Accordingly, in particular, the distal end or the distal portion of the electrode lead body is the end that is further distanced from the functional electrical stimulation device or the portion of the electrode lead body that is further distance from the functional electrical stimulation device.

The electrode lead can optionally comprise one or more electrodes for contacting bodily tissue, wherein the at least one electrode is arranged at the distal end of the electrode lead body, and in the case of a plurality of electrodes the plurality of electrodes are arranged distanced from one another in particular along the longitudinal axis. The electrodes are used to induce an electrical therapy at the body part to be treated, for example, in or at the heart, and/or to record measurement signals for diagnostic purposes. In addition to the unipolar stimulation, a bipolar stimulation of the tissue is also possible by a second electrode.

In one embodiment, the implantable electrode lead is also equipped with a third electrical conductor and a fourth electrical conductor, wherein the third electrical conductor and the fourth electrical conductor are arranged in at least one interior area of the electrode lead body. In addition, the electrode lead contains a further deformation sensor element arranged along the longitudinal axis of the electrode lead body, wherein the third electrical conductor and the fourth electrical conductor are conductively connected to the further deformation sensor element, and wherein the further deformation sensor element likewise comprises a biocompatible elastomer enriched with conductive particles. Due to the deformation sensor element arranged along the longitudinal axis of the electrode lead body and the further deformation sensor element arranged along the longitudinal axis of the electrode lead body, it is possible to detect deformations of the electrode lead in the region of the deformation sensor element and in the region of the further deformation sensor element at the same time.

In one embodiment, the elastomer of the deformation sensor element enriched with conductive particles is configured in the form of a tube. As a result of this embodiment, the deformation sensor element can be easily incorporated into the electrode lead or connected to the electrode lead and in specific embodiments can even take on specific functions of the electrode lead body.

In a further embodiment, the elastomer of the deformation sensor element enriched with conductive particles is configured in the form of a multi-lumen tube. In some electrode leads, the insulating part of the electrode lead body is formed by a multi-lumen tube, wherein this multi-lumen tube has a plurality of lumens for receiving electrical conductors and/or a guide wire and/or a mandrel. Here, the deformation sensor element can be formed by a portion of the multi-lumen tube, wherein this portion of the multi-lumen tube comprises an elastomer enriched with conductive particles.

In a further embodiment, the tribe forming the deformation sensor element surrounds the electrode lead body. This embodiment facilitates simple assembly of the deformation sensor element on the electrode lead.

In an alternative embodiment, the tube forming the deformation sensor element forms part of the outer casing of the electrode lead body. As a result, the diameter of the electrode lead can be kept constant in the region of the deformation sensor element.

In a further alternative embodiment, the tube forming the deformation sensor element forms part of the outer casing of the electrode lead body, wherein the tube for its part is coated at least partially or fully by a protective layer or by a protective casing. As a result of the protective casing, on the one hand the deformation sensor element is protected against external mechanical or chemical influences, and on the other hand the protective casing makes the deformation sensor element electrically insulated with respect to bodily fluids, for example, blood. During the measurement of the conductivity of the deformation sensor element, electrical charges are hereby prevented from flowing in or out via contact with the bodily fluid

In a further alternative embodiment, the elastomer of the deformation sensor element enriched with conductive particles is configured in the form of a bar. The bar in the region of the deformation sensor element is preferably arranged in an interior area of the electrode lead body in the form of a lumen. The deformation sensor element configured in the form of a bar can be introduced into a lumen of the electrode lead and can thus be situated in the interior of the electrode lead, where it is very well electrically insulated with respect to bodily fluids and is very well chemically shielded with respect to bodily fluids. The bar can further preferably also be arranged in a lumen not extending axially. If the bar is arranged in a lumen not extending axially, the bar is stretched or compressed to a greater extent in the event of bending movements of the electrode lead and therefore presents greater changes in electrical conductivity with the same deflection of the electrode lead. Smaller deformations, for example, deflections of the electrode lead, can hereby be detected.

In a further embodiment, the bar is configured in the form of a helix and the lumen in which the bar is arranged has a helical course along the axis of the electrode lead body. In the case of the embodiment of a helix, primarily torsional forces act on the bar of the deformation sensor element. The mechanical loading of the deformation sensor element can hereby be further reduced, which leads to a longer operating time and therefore a greater reliability of the deformation sensor element. The torsion of a bar formed from an elastomer enriched with conductive particles has an effect on the electrical conductivity of the bar. In particular, a bar subjected to torsional stress has a different electrical conductivity as compared to a bar not subjected to torsional stress.

In a further embodiment, the bar formed from elastomer enriched with conductive particles is arranged in the form of a helix around the lead body of the electrode lead. The bar can optionally also be embodied as a strip. Furthermore, the electrode lead body can have a groove, the course of which describes a helix on the surface of the electrode lead body, wherein the bar or the strip can be inserted into said groove.

In a further embodiment, the deformation sensor element has a first end and a second end, wherein the first electrical conductor is inductively connected to the first end of the deformation sensor element and the second electrical conductor is conductively connected to the second end of the deformation sensor element. The contacting of the deformation sensor element at both of its ends has the advantage that the entire length of the deformation sensor element is available for a measurement of the conductivity, and therefore the deformation is detectable over a maximum length along the longitudinal axis of the electrode lead in the region of the deformation sensor element.

In a further embodiment, the first electrical conductor is contacted with the deformation sensor element by a first annular electrode and the second electrical conductor is contacted with the deformation sensor element by a second annular electrode in that the first annular electrode surrounds the first end of the elastomer of the deformation sensor element enriched with conductive particles and the second annular electrode surrounds the second end of the elastomer of the deformation sensor element enriched with conductive particles, and wherein the first electrical conductor is electrically conductively connected to the first annular electrode and the second electrical conductor is electrically conductively connected to the second annular electrode. Good contacting of the elastomer of the deformation sensor element enriched with conductive particles is important for the accuracy and operational reliability of the deformation sensor element. The use of annular electrodes on the one hand offers a careful and on the other hand a large-area possibility of the contacting of the elastomer of the deformation sensor element enriched with conductive particles. The large contact area has the advantage that small local fluctuations in the density of the conductive particles enriched in the elastomer have no effect on the contacting quality. Furthermore, the electrical fields in the elastomer enriched with conductive particles for the measurement of the electrical conductivity can be constructed better and more uniformly by the annular electrodes. This leads to a better quality of the measurement and therefore to more accurate results in respect of the deformation.

In a further embodiment, the first annular electrode and/or the second annular electrode are/is surrounded by an insulating casing. The insulating casing prevents an outflow of charge from the annular electrodes into bodily fluids. The accuracy with which the deformation is detected is hereby increased.

In a further alternative embodiment, the elastomer of the deformation sensor element enriched with conductive particles is configured in the form of a first bar and a second bar, wherein the first bar and a second bar are arranged parallel to one another, and wherein the first bar is arranged in a first lumen of the electrode lead body and the second bar is arranged in a second lumen of the electrode lead body. More preferably, at least the first lumen, in which the first bar is arranged, or the second lumen, in which the second bar is arranged, is not arranged axially in the electrode lead body, and more preferably both aforesaid lumens are not arranged axially. These embodiments have the advantage that the deformation detection in respect of the bending movement is independent of the direction in which the electrode lead is bent at a specific point.

In a further embodiment, the first electrical conductor is electrically conductively connected to the first end of the deformation sensor element and the second electrical conductor is electrically conductively connected to the second end of the deformation sensor element. As a result of this embodiment, the first bar and the second bar are attached simultaneously to the first and the second electrical conductor. In particular, the first bar is thus connected at its first end to the first electrical conductor and the second bar is connected at its first end to the first electrical conductor, and furthermore the first bar is connected at its second end to the second electrical conductor and the second bar is connected at its second end to the second electrical conductor. The fail safety of the system is hereby increased. Furthermore, this embodiment offers the advantage that the detection of the deformation by the two bars is independent of the direction in which the electrode lead is bent by the force acting thereon.

In a further alternative embodiment, the first bar has a proximal end and a distal end and the second bar likewise has a proximal end and a distal end, wherein the distal end of the first bar is electrically conductively connected to the distal end of the second bar, and wherein the first electrical conductor is electrically conductively connected to the proximal end of the first bar and the second electrical conductor is electrically conductively connected to the proximal end of the second bar. As a result of this arrangement of the first bar and the second bar relative to one another and the contacting of the first bar with the second bar, the accuracy with which the deformation is detectable is increased.

In a further alternative embodiment, the elastomer of the deformation sensor element comprises a natural or synthetic rubber material, such as styrene-butadiene rubber (SBR) or acrylonitrile-butadiene rubber (AB), also referred to as nitrite rubber (NBR=nitrite butadiene rubber) for short, or silicone rubber, or a thermoplastic elastomer or liquid rubber. The elastomer of the deformation sensor element more preferably comprises silicone or polyurethane (PU) or polyether block amide block copolymer from the PEBAX® range by Arkema. These substances have proven their worth in the field of medical technology and are tried and tested and permitted for use within the body.

In a further embodiment, the conductive particles comprise conductive carbon, such as conductive carbon blacks or carbon fibers, metal-coated graphite (for example, coated with nickel) and metal-coated carbon (for example, coated with nickel), single-walled nanotubes (SWNT) or multi-walled nanotubes (MWNT) or nickel powder.

An elastomer to which nickel powder is added, preferably silicone or polyurethane, can form a QTC if the nickel particles have a porous, jagged surface with sharp edges. A suitable material for producing a QTC based on silicone or polyurethane is, for example, nickel powder of type T-123 or T-287 by Vale Inco Ltd. This powder for example has particle sizes of from 2 to 8 μm.

Additional features, aspects, objects, advantages, and possible applications of the present invention will become apparent from a study of the exemplary embodiments and examples described below, in combination with the Figures, and the appended claims.

### DESCRIPTION OF THE DRAWINGS

Further features, advantages and embodiments of the present invention will be described hereinafter with reference to the Figures, in which:

FIG. 1 shows an implantable electrode lead with a deformation sensor element;

FIG. 2 shows a portion of an implantable electrode lead in which the deformation sensor element is configured in the form of a tube;

FIG. 3 shows a portion of an implantable electrode lead in which the deformation sensor element is configured as a multi-lumen tube;

FIG. 4 shows a portion of a multi-lumen tube into which a deformation sensor element in the form of a bar is inserted;

FIG. 5 shows a portion of an implantable electrode lead which has a deformation sensor element in the form of a bar;

FIG. 6 shows a portion of an implantable electrode lead which has a deformation sensor element in the form of a wound bar; and

FIG. 7 shows a deformation sensor element connected to an implantable electrical stimulation device.

### DETAILED DESCRIPTION

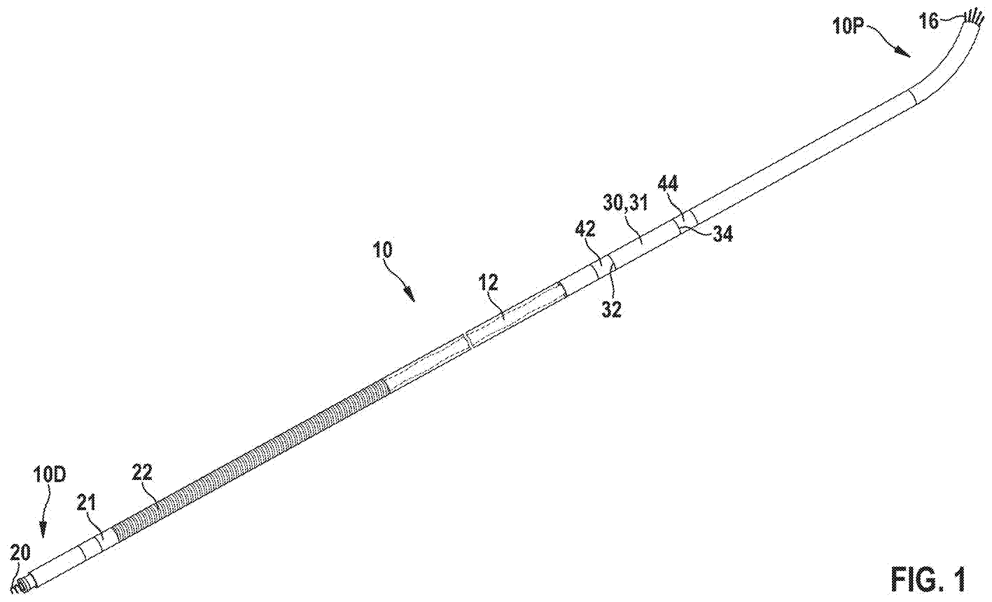

FIG. 1 shows a schematic view of an implantable electrode lead 10 with an elongate electrode lead body 12, which extends along a longitudinal axis of the implantable electrode lead 10. The implantable electrode lead 10 has at its distal end 10D a tip electrode 20, a ring electrode 21, and a shock coil 22 for contacting bodily tissue. The electrodes 20, 21 and the shock coil 22 are used to induce electrical therapy or to record measurement signals for diagnostic purposes. Furthermore, the implantable electrode lead 10 comprises a deformation sensor element 30 along its longitudinal axis of the electrode lead body 12. The deformation sensor element 30 of the implantable electrode lead 10 comprises a biocompatible elastomer 31 enriched with conductive particles.

For electrically conductive connection of the tip electrode 20, the ring electrode 21, the shock coil 22 and the first end 32 of the deformation sensor element 30 and the second end 34 of the deformation sensor element 30 to the proximal end 10P of the implantable electrode lead 10, electrical conductors 16 run in the interior of the electrode lead body 12. For improved contacting of the first end 32 and the second end 34 of the deformation sensor element 30 with one of the conductors 16, the implantable electrode lead 10 also has a first annular electrode 42 at the first end 32 of the deformation sensor element 30 and a second annular electrode 44 at the second end 34 of the deformation sensor element 30.

It should be noted at this juncture that the implantable electrode lead 10 shown by way of example in FIG. 1 has optional electrodes 20, 21 and 22. These optional electrodes 20, 21 and 22 are not necessary for the function of the deformation sensor element 30 described in FIG. 1. It is thus possible to produce an implantable electrode lead with deformation sensor element 30, which implantable electrode lead has arbitrary further electrodes in order to deliver signals to bodily tissue and/or record endogenous signals from the bodily tissue. Since one possible application of an implantable electrode lead 10 with deformation sensor element 30 is considered to lie in the field of the heart, an electrode lead 10 with deformation sensor element 30 which is embodied as an implantable stimulation and sensing electrode lead 10 for the cardiac region has been selected by way of example.

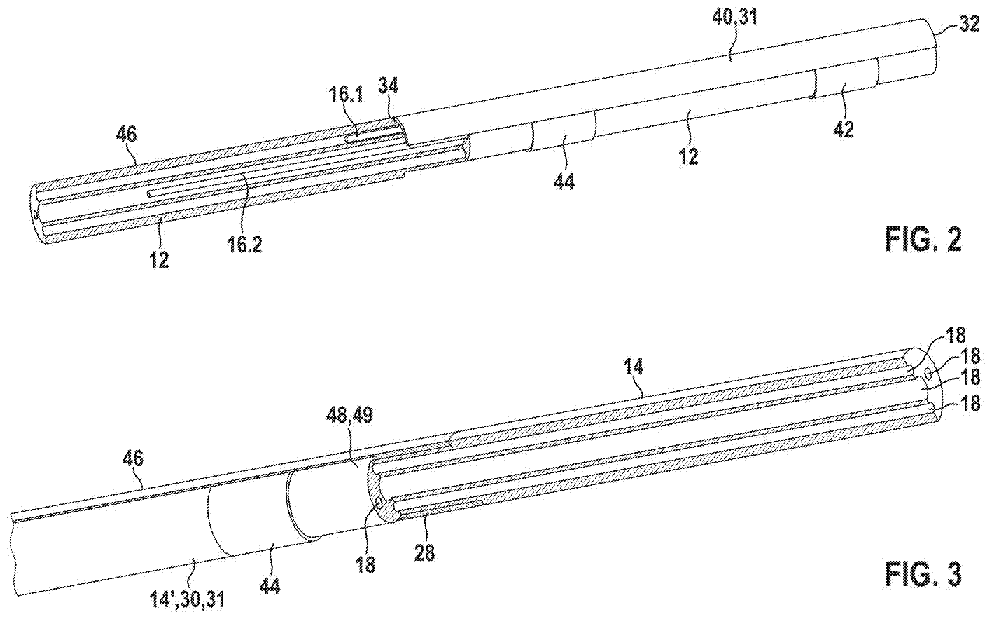

FIG. 2 shows a portion of an implantable electrode lead 10 with a deformation sensor element 30, wherein the elastomer 31 of the deformation sensor element 30 enriched with conductive particles is configured in the form of a tube 40. The tube 40 surrounds the electrode lead body 12. In FIG. 2 only the upper half of the tube 40 is shown, and therefore the encased electrode lead body 12 remains visible. Furthermore, FIG. 2 shows that the electrode lead body 12, outside the deformation sensor element 30, is surrounded by a (optional) protective layer 46. The deformation sensor element 30 formed as a tube 40 is contacted by the first annular electrode 42 arranged at the first end 32 of the deformation sensor element 30 and by the annular electrode 44 arranged at the second end 34 of the deformation sensor element 30. The deformation sensor element 30 is connected at its first end 32 to the first electrical conductor 16.1 by the first annular electrode 42, and the deformation sensor element 30 is connected at its second end 34 to the second electrical conductor 16.2 by the second annular electrode 44. In addition, the entire electrode lead 10 can be covered by an insulating casing 46 or a protective layer 46. Alternatively, only the region of the deformation sensor element 30 may be covered by a protective layer 46 or an insulating casing 46.

FIG. 3 shows a detailed portion of an implantable electrode lead 10 with a deformation sensor element 30, wherein the elastomer 31 of the deformation sensor element 30 enriched with conductive particles is configured in the form of a multi-lumen tube 14′. For improved clarity, the conductor 16 has not been shown in FIG. 3. In the shown electrode lead 10, the insulating part of the electrode lead body 12 is formed by a multi-lumen tube 14, wherein this multi-lumen tube 14 comprises a plurality of lumens 18 for receiving electrical conductors and/or a guide wire and/or a mandrel. Here, the deformation sensor element 30 can be formed by a portion of the multi-lumen tube 14′, wherein this portion of the multi-lumen tube 14′ comprises an elastomer 31 enriched with conductive particles.

FIG. 3 also shows a connection point 28 at which the multi-lumen tube 14′ forming the deformation sensor element 30 is adjacent to the multi-lumen tube 14 of the lead body. The elastomer of the multi-lumen tube 14 of the lead body is not enriched with conductive particles. Both the multi-lumen tube 14 of the lead body 12 and the deformation sensor element 30 are covered by an outer, insulating casing 46 in the form of a tube or a protective layer. A cavity 48 is provided at the connection point 28 between the deformation sensor element 30 and multi-lumen tube 14, and silicone adhesive 49 is filled into said cavity in order to glue the two elements. It is advantageous if silicone tubes are introduced into the lumens 18 at the connection point 28 in order to prevent the infiltration of silicone adhesive into the lumens 18 during the gluing process.

Alternatively, the deformation sensor element 30 embedded in the multi-lumen tube 14 can also be produced differently: during the extrusion of the multi-lumen tube 14, an elastomer 31 enriched with conductive particles can be used for the region forming the deformation sensor element 30, whereas an elastomer to which no conductive particles have been added is used for the regions extruded before and after the deformation sensor element 30.

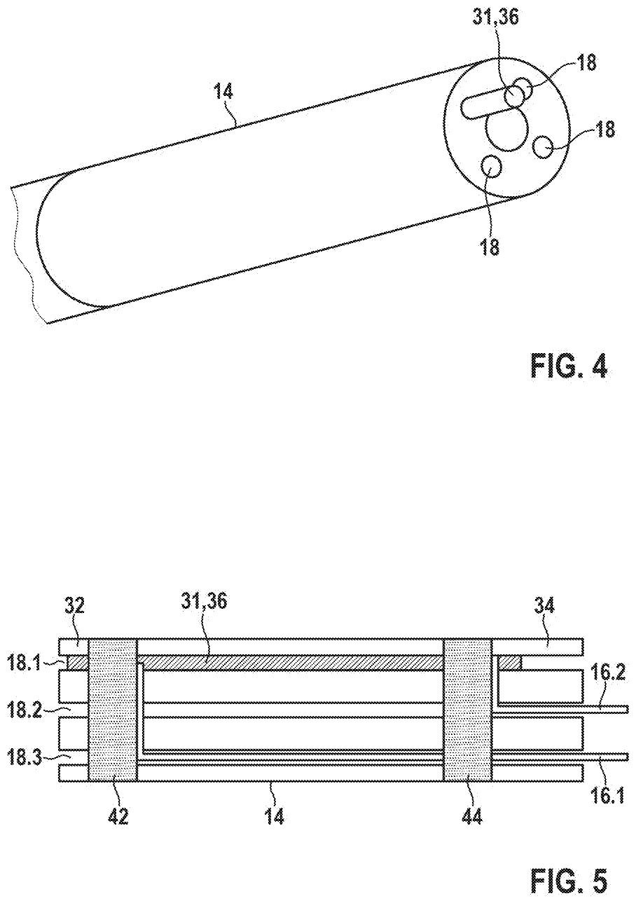

FIG. 4 shows a portion of a multi-lumen tube 14 with a central lumen 18 of greater diameter, for example for receiving a mandrel or a guide wire, and a plurality of non-axial lumens 18 of smaller diameter. A bar 36 formed from elastomer 31 enriched with conductive particles is inserted into one of the non-axial lumens 18.

FIG. 5 shows a longitudinal section through a portion of an electrode lead 10, wherein a bar 36 formed from elastomer 31 enriched with conductive particles is inserted into a first lumen 18.1 of the multi-lumen tube 14 of the electrode lead body 12 of the electrode lead 10. The bar 36 forms a deformation sensor element 30, which is surrounded at its first end 32 by a first annular electrode 42 and at its second end 34 by a second annular electrode 44. The deformation sensor element 30 is conductively connected at its first end 32 to the first electrical conductor 16.1 by the first annular electrode 42, and the deformation sensor element 30 is conductively connected at its second end 34 to the second electrical conductor 16.2 by the second annular electrode 44. The first electrical conductor 16.1 is then guided in the second lumen 18.2 to the proximal end 10P of the electrode lead 10, and the second electrical conductor 16.2 is guided in the third lumen 18.3 to the proximal end 10P of the electrode lead 10. Alternatively, it is also possible that the first electrical conductor 16.1 is guided in the first lumen 18.1 to the proximal end 10P of the electrode lead.

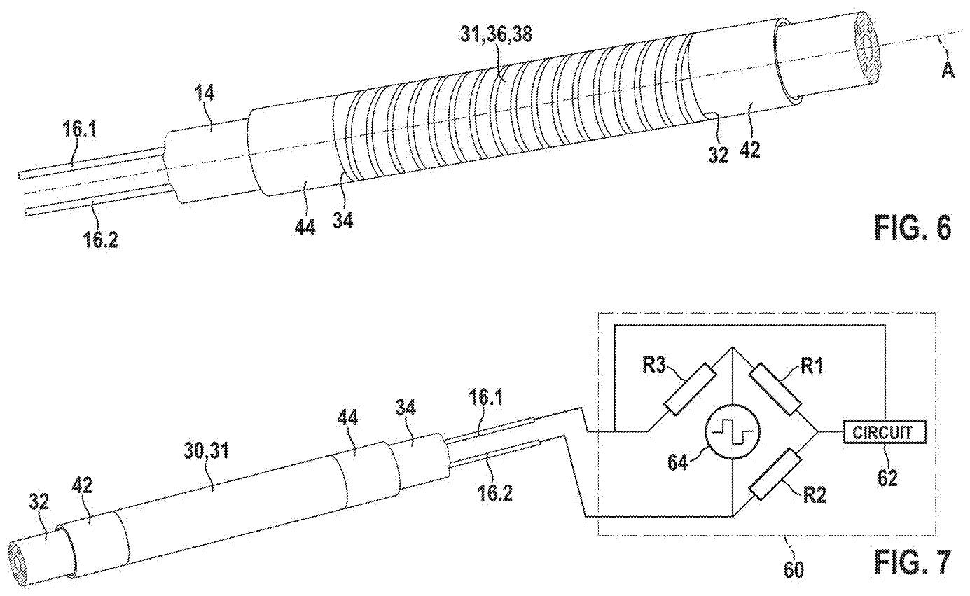

FIG. 6 shows a further possible construction for a deformation sensor element 30, in which a bar 36 formed from elastomer 31 enriched with conductive particles is placed in the form of a helix 38 around the lead body 12 of the electrode lead 10. The bar 36 may optionally also be embodied as a strip. The electrode lead body 12 can comprise a groove or channel (not shown), the course of which describes a helix 38 on the surface of the electrode lead body 12 in respect of the longitudinal axis A of the electrode lead body 12, wherein the bar 36 or the strip can be inserted into this groove. Alternatively, the bar 36 can also be inserted into a lumen (not shown), wherein the lumen in which the bar 36 is arranged has a helical course 38 along the longitudinal axis A of the electrode lead body 12.

The deformation sensor element 30 has a first annular electrode 42 at its first end 32 and has a second annular electrode 44 at its second end 34. The first annular electrode 42 connects the first end of the bar 36 or of the strip conductively to the first electrical conductor 16.1, and the second annular electrode 44 connects the second end of the bar 36 or of the strip conductively to the second electrical conductor 16.2. Although the bar 36 has always been shown in the drawings as a cylindrical bar 36, it may also have a different cross-sectional area, for example in the form of a polygon having 3, 4, 5, . . . n sides. The cross-sectional area can, in particular, also be rectangular or trapezoidal, diamond-shaped or in the form of a parallelogram. In this regard, the annular electrodes 42, 44 can have a form adapted to the cross-sectional area of the bar 36.

FIG. 7 shows a deformation sensor element 30 which is conductively connected to an implant 60 via a first electrical conductor 16.1 and via a second electrical conductor 16.2. In order for a deformation of the implantable electrode lead 10, for example, a change in the curvature, to be detected using the deformation sensor element 30, a continuous measurement of the impedance of the deformation sensor element 30 is necessary. For this purpose it can be proposed—as shown in FIG. 7—for the deformation sensor element 30 to be connected to an impedance-measuring unit 62 and a power source 64 by means of a bridge circuit. The further elements of the bridge circuit are resistors R1, R2 and R3. The deformation sensor element itself constitutes the fourth resistor in the bridge circuit. The power source 64 provides an alternating current, preferably a pulsed alternating current. If the deformations of the electrode lead that are to be measured lie in the region of the heart rate (0.5 to 4 Hz), the frequency of the fed alternating current should thus have frequencies in the range of from 5 Hz to 40 kHz.

It will be apparent to those skilled in the art that numerous modifications and variations of the described examples and embodiments are possible in light of the above teachings of the disclosure. The disclosed examples and embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

I/We claim:

1. An implantable electrode lead comprising:
an elongate electrode lead body;
a first electrical conductor and a second electrical conductor, wherein the first electrical conductor and the second electrical conductor are arranged in at least one interior area of the electrode lead body; and
a deformation sensor element arranged along the electrode lead body, wherein the first electrical conductor and the second electrical conductor are conductively connected to the deformation sensor element, and wherein the deformation sensor element comprises a biocompatible elastomer enriched with conductive particles.

2. The implantable electrode lead according to claim 1, wherein the elastomer of the deformation sensor element enriched with conductive particles is provided in the form of a tube.

3. The implantable electrode lead according to claim 2, wherein the tube:
surrounds the electrode lead body, or
forms part of the outer casing of the electrode lead body.

4. The implantable electrode lead according to claim 1, wherein the elastomer of the deformation sensor element enriched with conductive particles is provided in the form of a bar.

5. The implantable electrode lead according to claim 4, wherein the interior area of the electrode lead body is formed as a lumen, and wherein the bar is arranged in the lumen of the electrode lead body in the region of the deformation sensor element.

6. The implantable electrode lead according to claim 5, wherein the bar is provided in the form of a helix, and wherein the lumen has a helical course along the axis of the electrode lead body.

7. The implantable electrode lead according to claim 2, wherein the deformation sensor element has a first end and a second end, wherein the first electrical conductor is conductively connected to the first end of the deformation sensor element, and wherein the second electrical conductor is conductively connected to the second end of the deformation sensor element.

8. The implantable electrode lead according to claim 7, wherein the first electrical conductor is contacted with the deformation sensor element by a first annular electrode and the second electrical conductor is contacted with the deformation sensor element by a second annular electrode in that the first annular electrode surrounds the first end of the elastomer of the deformation sensor element enriched with conductive particles and the second annular electrode surrounds the second end of the elastomer of the deformation sensor element enriched with conductive particles, and wherein the first electrical conductor is electrically conductively connected to the first annular electrode and the second electrical conductor is electrically conductively connected to the second annular electrode.

9. The implantable electrode lead according to claim 8, wherein the first annular electrode and/or the second annular electrode are/is surrounded by an insulating casing.

10. The implantable electrode lead according to claim 1, wherein the elastomer of the deformation sensor element enriched with conductive particles is provided in the form of a first bar and a second bar, wherein the first bar and the second bar are arranged parallel to one another, and wherein the first bar is arranged in a first lumen of the electrode lead body and the second bar is arranged in a second lumen of the electrode lead body.

11. The implantable electrode lead according to claim 10, wherein the first electrical conductor is electrically conductively connected to a first end of the deformation sensor element, and the second electrical conductor is electrically conductively connected to a second end of the deformation sensor element.

12. The implantable electrode lead according to claim 10, wherein the first bar has a proximal end and a distal end and the second bar has a proximal end and a distal end, wherein the distal end of the first bar is electrically conductively connected to the distal end of the second bar, and wherein the first electrical conductor is electrically conductively connected to the proximal end of the first bar and the second electrical conductor is electrically conductively connected to the proximal end of the second bar.

13. The implantable electrode lead according to claim 1, wherein the elastomer of the deformation sensor element comprises one of the following materials: styrene-butadiene rubber (SBR) or acrylonitrile-butadiene rubber (AB), or silicone rubber, or a thermoplastic elastomer or liquid rubber or silicone or polyurethane or a polyether block amide block copolymer.

14. The implantable electrode lead according to claim 1, wherein the conductive particles comprise conductive carbon black, carbon fibers, metal-coated graphite, metal-coated carbon, single-walled nanotubes (SWNT), multi-walled nanotubes (MWNT) or nickel powder.

15. The implantable electrode lead according to any one of the preceding claims, wherein a volume fraction of the conductive particles in the elastomer provided with conductive particles is between 0.8 vol. % and 45 vol. %.
